(19) United States
(12) Patent Application Publication
(10) Pub. No.: US 2008/0064681 A1
(43) Pub. Date: Mar. 13, 2008

(54) Therapeutic agent for treating glaucoma

(76) Inventors: Hiroyoshi Hidaka, Nagoya-shi (JP); Masahiro Nishio, Tsu-shi (JP); Kengo Sumi, Seki-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/518,131

(22) Filed: Sep. 11, 2006

(51) Int. Cl. A61K 31/551; A61K 31/496; A61K 31/4709

(52) U.S. Cl. 514/218; 514/253.06; 514/307

(57) ABSTRACT

The present invention provides compounds useful as therapeutic agent for treating glaucoma and methods for treating glaucoma. That is, a therapeutic agent for treating glaucoma containing, as an active ingredient, a compound represented by formula (1) below is synthesized, and the therapeutic agent is administered in the form of eye drops to a glaucoma patient. Thus, the intraocular pressure is reduced.

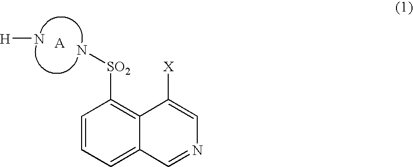

In the above formula, ring A represents a 5- to 11-membered cyclic amino group, which may have a substituted group, and X represents a halogen.

### TECHNICAL FIELD

The present invention relates to compounds useful as therapeutic agents for treating glaucoma and drugs containing the same.

### BACKGROUND ART

Glaucoma is an eye disease in which the field of vision gradually narrows without any warning signs, such as prodromal symptoms. Increased pressure in the eyeball (intraocular pressure) caused by glaucoma damages the optic nerve, and results in loss of vision as a terminal symptom. As therapeutic agents for treating glaucoma, a prostaglandin analogue Xalatan (Ophthalmology 103 (1996): 138; Ophthalmology 103 (1996): 126; J. Ophthalmol. 132 (2001): 472) and the like are known.

With respect to other pharmaceutical agents, it has been reported that Rho-kinase inhibitors, such as Y-27632 and HA-1077, reduce intraocular pressure in rabbits (Invest. Ophthalmol. Visual Sci. 42 (2001): 137; Arch. Opthalmol. 119 (2001): 1171; Invest. Ophthalmol. Visual Sci. 42 (2001): 1029). Y-27632 is widely used, mainly in biochemical experiments and pharmacological experiments, and HA-1077 is widely used, as Eril, for treating cerebral vasospasm after subarachnoid hemorrhage surgery. Furthermore, it has been reported that a compound obtained by substituting the methyl group at position 4 of the isoquinoline ring in H-1152P, which has a similar structure to HA-1077, with an ethynyl group exhibits intraocular-pressure-reducing activity (Biochimi. Biophys. Acta 1754 (2005): 245). However, with respect to other isoquinoline derivatives, their cerebral vasospasm-inhibiting activity has only been shown (Japanese Unexamined Patent Application Publication No. 11-349482), but intraocular-pressure-reducing activity has not been disclosed at all.

Although Rho-kinase inhibitors are thus considered to be promising new therapeutic agents for treating glaucoma, it has not been clear whether or not the compound having Rho-kinase inhibitory activity in vitro actually has intraocular-pressure-reducing activity in vivo.

Accordingly, the present invention has been achieved for the purpose of providing a compound useful as a therapeutic agent for treating glaucoma.

### DISCLOSURE OF INVENTION

The present inventors have examined the relationship between Rho-kinase inhibitory activity in vitro and intraocular-pressure-reducing activity in vivo for compounds having Rho-kinase inhibitory activity. For example, for compounds obtained by substituting the methyl group at position 4 of the isoquinoline ring of H-1152P with fluorine, bromine, chlorine, or an ethynyl group, only the intraocular-pressure-reducing activity of the ethynyl group-substituted derivative has been reported (Biochimi. Biophys. Acta 1754 (2005): 245). Thus, intraocular-pressure-reducing activity has been examined for various compounds having Rho-kinase inhibitory activity including the compounds described above. As a result, the inventors has found that all compounds having Rho-kinase inhibitory activity do not have intraocular-pressure-reducing activity, and that compounds obtained by substituting the methyl group at position 4 of the isoquinoline ring of H-1152P with a halogen and 2-(S)-octahydro-1-(4-haloisoquinoline-5-sulfonyl)-2-methyl-1,4-diazocine 2 have intraocular-pressure-reducing activities with different characteristics. The present invention has thus been completed.

That is, according to an aspect of the present invention, a method for treating glaucoma is provided, which includes administering to a glaucoma patient a compound represented by any one of formulae (1) to (5):

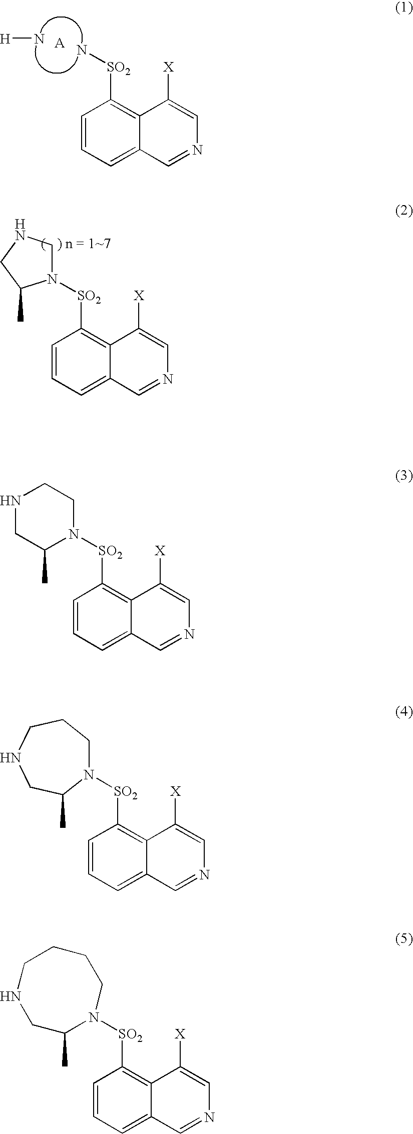

wherein ring A represents a 5- to 11-membered cyclic amino group, which may be substituted, and X represents a halogen).

As the method of administration, preferably, eye drops containing the compound described above are administered to a glaucoma patient. Thus, it is possible to reduce the intraocular pressure of the glaucoma patient.

### BRIEF DESCRIPTION OF THE DRAWINGS

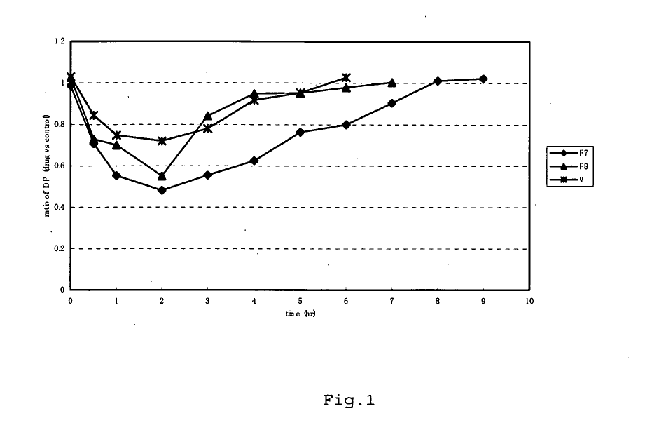

FIG. 1 is a graph showing the change in intraocular pressure with time in an example, in which 1% phosphate buffer solution of
(S)-hexahydro-1-(4-fluoroisoquinoline-5-sulfonyl)-2-methyl-1H-1,4-diazepine dihydrochloride (F7),
(S)-octahydro-1-(4-fluoroisoquinoline-5-sulfonyl)-2-methyl-1,4-diazocine dihydrochloride (F8), or
(S)-hexahydro-1-(4-ethynylisoquinoline-5-sulfonyl)-2-methyl-1H-1,4-diazepine dihydrochloride (reference compound) was instilled into the right eye, phosphate buffer as control was instilled into the left eye, their intraocular pressures were measured serially, and the ratios to the control values were calculated and plotted.

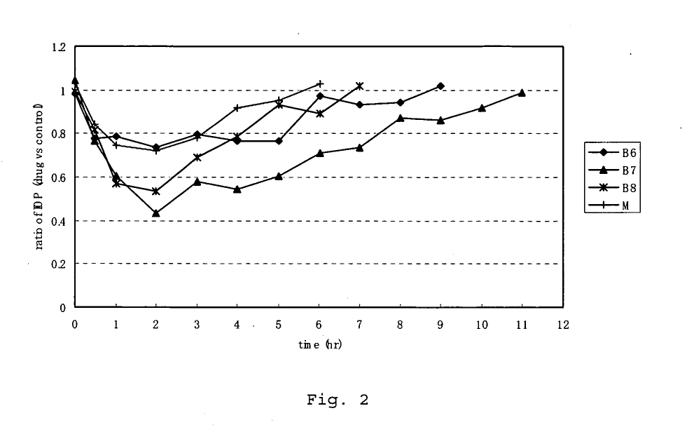

FIG. 2 is a graph showing the change in intraocular pressure with time in the example, in which 1% phosphate buffer solution of (S)-1-(4-bromoisoquinoline-5-sulfonyl)-2-methylpiperazine dihydrochloride (B6),
(S)-hexahydro-1-(4-bromoisoquinoline-5-sulfonyl)-2-methyl-1H-1,4-diazepine dihydrochloride (B7),
(S)-octahydro-1-(4-bromoisoquinoline-5-sulfonyl)-2-methyl-1,4-diazocine dihydrochloride (B8), or
(S)-hexahydro-1-(4-ethynylisoquinoline-5-sulfonyl)-2-methyl-1H-1,4-diazepine dihydrochloride (reference compound) was instilled into the right eye, phosphate buffer as control was instilled into the left eye, their intraocular pressures were measured serially, and the ratios to the control values were calculated and plotted.

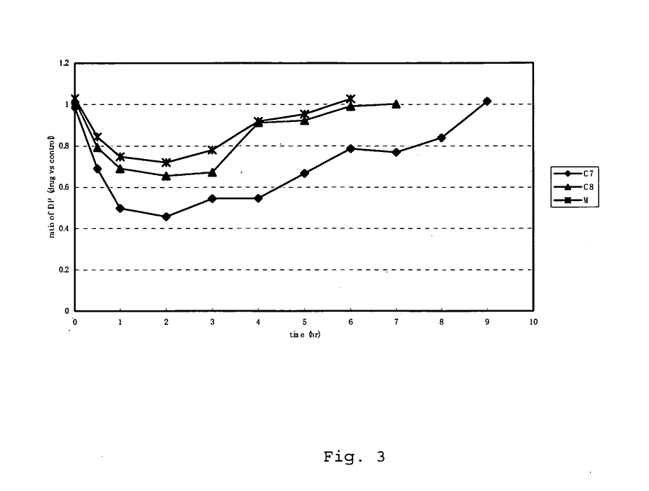

FIG. 3 is a graph showing the change in intraocular pressure with time in the example, in which 1% phosphate buffer solution of
(S)-hexahydro-1-(4-chloroisoquinoline-5-sulfonyl)-2-methyl-1H-1,4-diazepine dihydrochloride (C7),
(S)-octahydro-1-(4-chloroisoquinoline-5-sulfonyl)-2-methyl-1,4-diazocine dihydrochloride (C8), or
(S)-hexahydro-1-(4-ethynylisoquinoline-5-sulfonyl)-2-methyl-1 H-1,4-diazepine dihydrochloride (reference compound) was instilled into the right eye, phosphate buffer as control was instilled into the left eye, their intraocular pressures were measured serially, and the ratios to the control values were calculated and plotted.

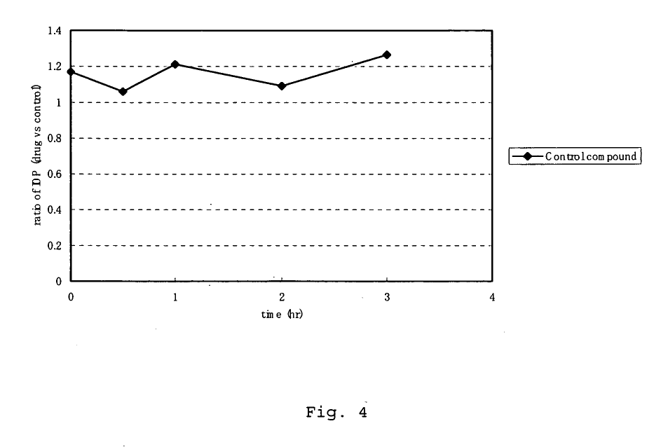

FIG. 4 is a graph showing the change in intraocular pressure with time in an example, in which 1% phosphate buffer solution of
(S)-hexahydro-1-(4-fluoroisoquinoline-5-sulfonyl)-4-glycyl-2-methyl-1H-1,4-diazepine dihydrochloride was instilled into the right eye, phosphate buffer as control was instilled into the left eye, their intraocular pressures were measured serially, and the ratios to the control values were calculated and plotted.

### DETAILED DESCRIPTION

### ==4-Haloisoquinoline Derivatives==

A therapeutic agent for treating glaucoma according the present invention contains, as an active ingredient, a compound represented by formula (1):

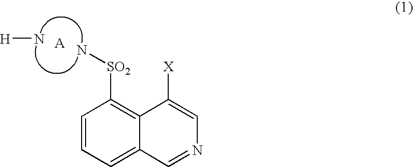

wherein ring A represents a 5- to 11-membered cyclic amino group, which may be substituted, and X represents a halogen, namely, a compound having a cyclic aminosulfonyl group at position 5 of 4-haloisoquinoline. As will be shown in examples, the intraocular-pressure-reducing activity is not influenced by the size of the ring of the cyclic aminosulfonyl group. Consequently, the cyclic aminosulfonyl group may be any one of 5- to 11-membered rings. Furthermore, in the cyclic aminosulfonyl group, the side chain bonded to the carbon is preferably hydrogen or C1 to C4 alkyl.

In particular, the therapeutic agent for treating glaucoma according to the present invention is preferably a compound represented by formula (2):

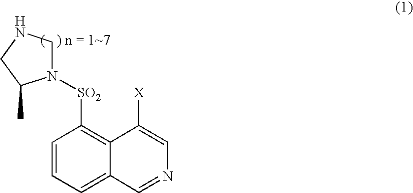

wherein X represents a halogen.

Furthermore, the therapeutic agent for treating glaucoma according to the present invention is more preferably a compound represented by any one of formulae (3) to (5):

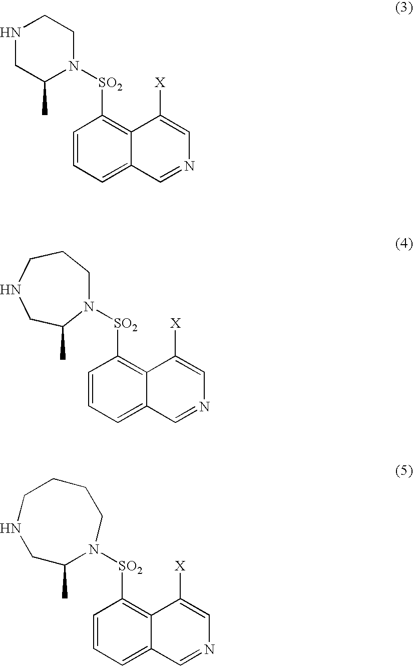

wherein X represents a halogen.

Furthermore, in particular, the therapeutic agent for treating glaucoma according to the present invention is more preferably a compound represented by any one of formulae (6) to (8):

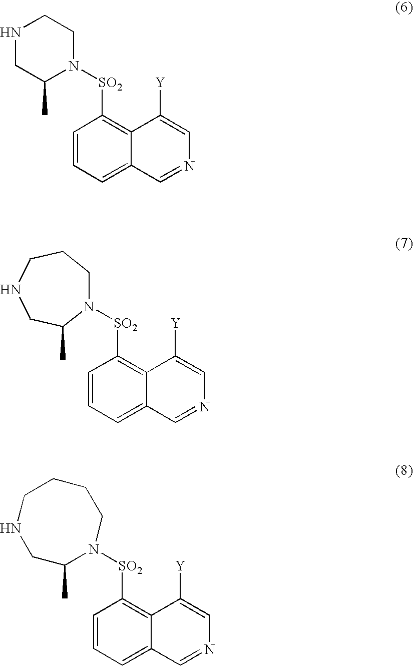

wherein Y represents any one of Br, Cl, and F.

These compounds can be present in the form of a salt or in the form of a solvate such as a hydrate. As the salt, any pharmaceutically acceptable salt can be used without limitations. Their examples include salts of mineral acids, such as hydrochlorides, hydrobromides, hydroiodides, sulfates, and phosphates; and salts of organic acids, such as benzoates, methanesulfonates, ethanesulfonates, benzenesulfonates, benzenesulfonates, p-toluenesulfonates, oxalates, maleates, fumarates, tartrates, citrates, and acetates.

### ==Method for Producing 4-haloisoquinoline Derivatives==

The compound (1) can be produced according to a known method which is described in International Publication No. WO97/28130.

For example, the compound (2) can be produced by the method shown below.

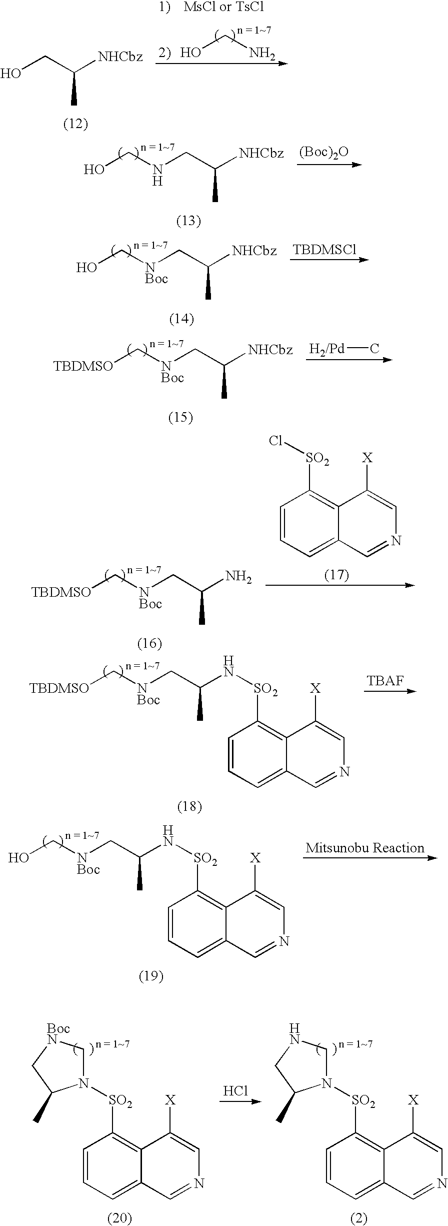

The compound (12), which is a starting material for the compound of the present invention, is commercially available from Aldrich or the like. The reaction for obtaining the compound (13) from the compound (12) is carried out by converting the hydroxyl group of the compound (12) into methanesulfonyloxy, tosyloxy, or the like by a known method, and then allowing it to react with an aminoalkyl alcohol (e.g., 4-amino-1-butanol) in an appropriate solvent.

The conversion reaction into methanesulfonyloxy or tosyloxy is carried out by allowing the compound (12) to react with methanesulfonyl chloride or the like in the presence of a tertiary amine, such as triethylamine. Examples of the reaction solvent used for the subsequent reaction with the aminoalkyl alcohol (e.g., 4-amino-1-butanol) include halogenated hydrocarbons, such as dichloromethane and chloroform; ethers, such as tetrahydrofuran (THF) and diethyl ether; N,N-dimethylformamide (DMF), acetonitrile, and the like. The reaction is carried out at 0° C. to around the boiling point of the solvent for 1 to 48 hours, preferably at 40° C. to 100° C. for 2 to 12 hours.

The amount of the aminoalkyl alcohol (e.g., 4-amino-1-butanol) used is 1 to 10 equivalents, preferably 4 to 6 equivalents, relative to the compound (12).

The amine of the resulting compound (13) is protected with a protecting group, such as a tert-butoxycarbonyl group, a formyl group, or a benzoyl group, to give the compound (14). The hydroxyl group is then protected with a protecting group, such as a tert-butyldimethylsilyl group or an acetyl group, to give the compound (15). The hydroxyl group-protecting reaction may be omitted in some cases. The benzyloxycarbonyl group of the compound (15) is removed by hydrogenation in the presence of a metal catalyst, such as palladium, to give the compound (16). As the protecting group, tert-butoxycarbonyl is preferable.

The amino group-protecting reaction is carried out by allowing the compound (13) to react with the tert-butoxycarbonyl group or the like in the presence of a tertiary amine, such as triethylamine. The removal reaction of the protecting group is carried out by adding hydrogen in an alcohol solvent in the presence of palladium-carbon. The protecting reaction of the hydroxyl group is carried out in the presence of a tertiary amine using a protecting group, such as a tert-butyldimethylsilyl group or an acetyl group.

The reaction between the primary amine (16) and the compound (17) is carried out in an appropriate solvent, preferably, in the presence of a necessary amount of a base. Examples of the base include inorganic bases, such as potassium carbonate, sodium carbonate, and cesium carbonate; and organic bases, such as triethylamine, diisopropylamine, and triethyldiamine. The reaction solvent is the same as that used for obtaining the compound (13). The reaction is carried out at 0° C. to 80° C. for 0.5 to 24 hours, preferably at 10° C. to 50° C. for 1 to 8 hours.

The primary amine (16) is preferably used in an amount of 1 to 3 equivalents relative to the compound (17). It should be noted that the compound (17) can be synthesized by the method described in Japanese Unexamined Patent Application Publication No. 2-67274 or by an analogous method.

Deprotection of the hydroxyl group of the resulting compound (18) is performed, for example, using tetrabutylammonium fluoride or the like in the case of protection with the tert-butyldimethylsilyl group, to give the compound (19). The resulting compound (19) is subjected to ring closure, for example, by the Mitsunobu reaction using triphenylphosphine and an azodicarboxylate ester or the like to give the compound (20). The deprotection reaction of the compound (20) is carried out by a known method suitable for the protecting group, for example, by acid treatment, alkaline treatment, or catalytic reduction. For example, when the compound (20) is protected with the tert-butoxycarbonyl group, the compound (2) of the present invention can be obtained by treatment with a 1,4-dioxane solution or an ethyl acetate solution of hydrogen chloride, or the like.

The compound (2) of the present invention can be obtained by the method described above. Furthermore, the compound (2) can be optionally purified using a known purification technique, such as recrystallization or column chromatography. The compound (2) can be optionally converted into a desired salt or solvate by a known method.

### ==Form of Therapeutic Agent for Treating Glaucoma==

As shown in Examples below, the compounds (1) to (8), ζ their salts, or their solvates can reduce intraocular pressure in glaucoma animal models, and thus are useful as drugs for treating glaucoma.

The therapeutic agent for treating glaucoma containing any of the compounds (1) to (8), their salts, or their solvates is preferably administered in the form of eye drops. The eye drops are prepared by blending a pharmaceutically acceptable carrier using a preparation method known to persons skilled in the art.

While the dose of the drug of the present invention varies depending on the age, body weight, symptom, administration form, number of doses, and the like, it is usually preferred that the drug is administered as eye drops one or several times in a dose of 1 to 1,000 mg per day for an adult.

### EXAMPLES

The present invention will be described in detail below with reference to the examples, to which the present invention is not limited.

### Example 1

### Synthesis of Compounds

### Synthesis of (S)-hexahydro-1-(4-fluoroisoquinoline-5-sulfonyl)-2-methyl-1H-1,4-diazepine dihydrochloride (hereinafter referred to as compound F7)

This compound was synthesized specifically according to the conditions described in International Publication No. WO99/20620.

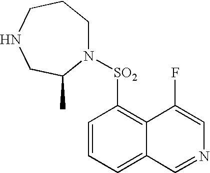

### Synthesis of (S)-octahydro-1-(4-fluoroisoquinoline-5-sulfonyl)-2-methyl-1,4-diazocine dihydrochloride (hereinafter referred to as compound F8)

### (1) Synthesis of 2-(S)-2-(benzyloxycarbonyl)amino-N-(tert-butoxycarbonyl)-N-(4-hydroxybutyl)propylamine

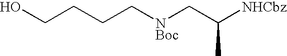

2-(S)-2-(Benzyloxycarbonyl)amino-1-propanol (2.10 g, 10.0 mmol) was dissolved in chloroform (21 mL), and the resulting solution was cooled down to 0° C. Triethylamine (2.08 mL, 15.0 mmol) and methane sulfonyl chloride (1.16 mL, 15.0 mmol) were added to the solution, and the mixture was stirred at room temperature for one hour. Subsequently, distilled water was added to the solution, which was then extracted with chloroform. The organic layer was washed with saturated brine and dried over anhydrous sodium sulfate, followed by vacuum concentration. The resulting crystals were dried to give a crude product (3.00 g). Subsequently, the crude product (1.50 g) was weighed and dissolved in THF (10 mL). 4-Amino-1-butanol (2.22 g, 24.9 mmol) was added to the resulting solution, which was then stirred at 80° C. for 12 hours. After the mixture was brought back to room temperature, distilled water was added to the mixture, which was extracted with dichloromethane. The organic layer was washed with distilled water and dried over anhydrous sodium sulfate, followed by vacuum concentration. Then, the residue was dissolved in dichloromethane (10 mL) and the resulting solution was cooled down to 0° C. Triethylamine (835 μL, 6.00 mmol) and di-tert-butyl dicarbonate (1.31 g, 6.00 mmol) were added to the solution, and the mixture was stirred at room temperature for one hour. Distilled water was added to the solution, which was then extracted with ethyl acetate. The organic layer was dried over anhydrous sodium sulfate, followed by vacuum concentration. The residue was purified by silica gel column chromatography (developing solvent: n-hexane/ethyl acetate=4/5 to 1/2) to give the aimed compound as a light yellow oily substance (yield: 809 mg, 2.12 mmol, 42.5%, 3 steps). TLC Rf=0.23 (n-hexane/ethyl acetate=1/1)

### (2) Synthesis of 2-(S)-2-(benzyloxycarbonyl)amino-N-(tert-butoxycarbonyl)-N-(4-tert-butyldimethylsilyloxybutyl)propylamine

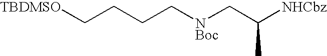

2-(S)-2-(Benzyloxycarbonyl)amino-N-(tert-butoxycarbonyl)-N-(4-hydroxybutyl)propylamine (1.05 g, 2.75 mmol) was dissolved in dichloromethane (5 mL), and imidazole (300 mg, 4.40 mmol) and tert-butyldimethylsilyl chloride (678 mg, 4.49 mmol) were added to the solution, followed by stirring at room temperature for 12 hours. A saturated aqueous ammonium chloride solution was added to the solution, which was then extracted with ethyl acetate. The organic layer was washed with a saturated aqueous ammonium chloride solution, while the aqueous layer was extracted with ethyl acetate. The organic layers were combined and were dried over anhydrous sodium sulfate, followed by vacuum concentration. The residue was purified by silica gel column chromatography (developing solvent: n-hexane/ethyl acetate=1/1) to give the aimed compound as a colorless oily substance (yield: 1.29 g, 2.61 mmol, 94.9%). TLC Rf=0.76 (n-hexane/ethyl acetate=1/1)

### (3) Synthesis of 2-(S)-2-amino-N-(tert-butoxycarbonyl)-N-(4-tert-butyldimethyl silyloxybutyl)propylamine

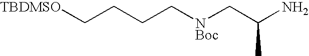

2-(S)-2-(Benzyloxycarbonyl)amino-N-(tert-butoxycarbonyl)-N-(4-tert-butyldimethylsilyloxybutyl)propylamine (1.29 g, 2.61 mmol) was dissolved in methanol (5 mL). Catalytic reduction was carried out in the presence of 10% palladium-activated carbon (129 mg) under hydrogen flow. After completion of the reaction, dichloromethane was added to the mixture, which was then filtered with Celite to remove the catalyst. The filtrate was concentrated under vacuum to give the aimed compound as a colorless oily substance (yield: 875 mg). This substance was used in the subsequent reaction directly without purification.

### (4) Synthesis of 2-(S)-1-(4-fluoroisoquinoline-5-sulfonyl)-N-(tert-butoxycarbonyl)-N-(4-tert-butyldimethylsilyloxybutyl)propylamine

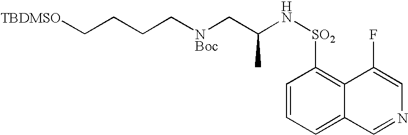

2-(S)-2-Amino-N-(tert-butoxycarbonyl)-N-(4-tert-butyldimethylsilyloxybutyl)propylamine (393 mg, 1.08 mmol) was dissolved in dichloromethane (3 mL), and the resulting solution was cooled down to 0° C. Triethylamine (150 μL, 1.08 mmol), a catalytic amount of 4-dimethylaminopyridine, and 4-fluoroisoquinoline-5-sulfonyl chloride (245 mg, 1.00 mmol) were subsequently added to the solution, which was then stirred at room temperature for 16 hours. Distilled water was added to the solution, which was then extracted with dichloromethane. The organic layer was washed with distilled water and dried over anhydrous sodium sulfate, followed by vacuum concentration. The residue was purified by silica gel column chromatography (n-hexane/acetone=4/1 to 3/1) to give the aimed compound as a colorless oily substance (yield: 381 mg, 0.668 mmol, 66.8%). TLC Rf=0.33 (n-hexane/acetone=3/1)

### (5) Synthesis of 2-(S)-1-(4-fluoroisoquinoline-5-sulfonyl)-N-(tert-butoxycarbonyl)-N-(4-hydroxybutyl)propylamine

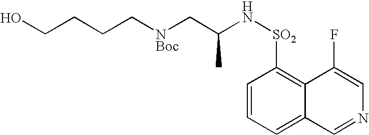

2-(S)-1-(4-Fluoroisoquinoline-5-sulfonyl)-N-(tert-butoxycarbonyl)-N-(4-tert-butyldimethylsilyloxybutyl)propylamine (381 mg, 0.688 mmol) was dissolved in anhydrous THF (5 mL), and 1.0 M tetrabutylammonium fluoride (1.00 mL, 1.00 mmol) was added to the solution, which was the stirred at room temperature for 24 hours. A saturated aqueous ammonium chloride solution and dichloromethane were added to the solution, and the organic layer was washed with saturated ammonium chloride three times. The aqueous layer was then extracted with dichloromethane, and the organic layer was dried over anhydrous sodium sulfate, followed by vacuum concentration. The residue was purified by silica gel column chromatography (dichloromethane/methanol=30/1 to 25/1) to give the aimed compound as a colorless foam (yield: 280 mg, 0.614 mmol, 92.0%). TLC Rf=0.28 (dichloromethane/methanol=9/1)

### (6) Synthesis of (S)-octahydro-4-(tert-butoxycarbonyl)-1-(4-fluoroisoquinoline-5-sulfonyl)-2-methyl-1,4-diazocine

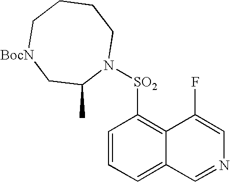

Under an argon atmosphere, 2-(S)-1-(4-fluoroisoquinoline-5-sulfonyl)-N-(tert-butoxycarbonyl)-N-(4-hydroxybutyl)propylamine (280 mg, 0.614 mmol) was dissolved in anhydrous THF (5 mL). Triphenylphosphine (322 mg, 1.22 mmol) and diisopropyl azodicarbonate (240 μL, 1.22 mmol) were added to the solution, followed by stirring at room temperature for 18 hours. Subsequently, vacuum concentration was performed, and the residue was purified by silica gel column chromatography (n-hexane/acetone=3/1 to 5/2) to give the aimed compound as a crude product in a white foam (yield: 262 mg, 0.598 mmol, 97.5%). TLC Rf=0.40 (n-hexane/acetone=2/3)

### (7) Synthesis of (S)-octahydro-1-(4-fluoroisoquinoline-5-sulfonyl)-2-methyl-1,4-diazocine dihydrochloride

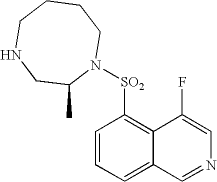

(S)-Octahydro-4-(tert-butoxycarbonyl)-1-(4-fluoroisoquinoline-5-sulfonyl)-2-methyl-1,4-diazocine (126 mg, 0.287 mmol) was dissolved in chloroform (1 mL), and a 1,4-dioxane solution of 4 M hydrochloric acid (2 mL, 8.00 mmol) was added to the solution. After completion of the reaction, precipitated crystals were collected and washed with dichloromethane on a funnel to give the aimed compound as a light yellow solid. (85.3 mg, 0.207 mmol, 72.4%, 2 steps)

¹H-NMR (400 MHz, DMSO-d₆) d 0.61 (d, 3H, J=6.4 Hz, CH₃), 1.36-1.64 (m, 4H, 2CH₂), 2.77-3.08 (m, 4H, 2CH₂), 3.22-3.29 (m, 1H, CH), 3.37-3.41 (m, 1H, CH), 3.87-3.92 (m, 1H, CH), 7.65 (t, 1H, J=8.0 Hz, aromatic), 8.16 (d, 1H, J=8.0 Hz, aromatic), 8.28 (d, 1H, J=8.0 Hz, aromatic), 8.41 (d, 1H, J=4.8 Hz aromatic), 9.08 (s, 1H, aromatic).

### Synthesis of 2-(S)-1-(4-bromoisoquinoline-5-sulfonyl)-2-methylpiperazine dihydrochloride (hereinafter referred to as compound B6)

### (1) Synthesis of 2-(S)-2-(benzyloxycarbonyl)amino-N-(tert-butoxycarbonyl)-N-(2-hydroxyethyl)propylamine

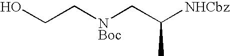

2-(S)-2-(Benzyloxycarbonyl)amino-1-propanol (2.99 g, 14.2 mmol) was dissolved in chloroform (30 mL), and the resulting solution was cooled down to 0° C. Triethylamine (3.0 mL, 21.5 mmol) and methane sulfonyl chloride (1.66 mL, 21.5 mmol) were added to the solution, and the mixture was stirred at room temperature for one hour. Subsequently, distilled water was added to the solution, which was then extracted with chloroform. The organic layer was washed with saturated brine and dried over anhydrous sodium sulfate, followed by vacuum concentration. The resulting crystals were dried to give a crude product (4.70 g). Subsequently, the crude product (750 mg) was weighed and dissolved in THF (5 mL). 2-Amino-1-ethanol (763 mg, 12.5 mmol) was added to the resulting solution, which was then stirred at 90° C. for 12 hours. After the mixture was brought back to room temperature, distilled water was added to the solution, which was then extracted with dichloromethane. The organic layer was washed with distilled water and dried over anhydrous sodium sulfate, followed by vacuum concentration. The residue was then dissolved in dichloromethane (5 mL) and the resulting solution was cooled down to 0° C. Triethylamine (420 μL, 3.01 mmol) and di-tert-butyl dicarbonate (657 mg, 3.01 mmol) were added to the solution, and the mixture was stirred at room temperature for 18 hours. Distilled water was added to the solution, which was then extracted with ethyl acetate. The organic layer was dried over anhydrous sodium sulfate, followed by vacuum concentration. The residue was purified by silica gel column chromatography (developing solvent: n-hexane/ethyl acetate=4/5 to 2/3) to give the aimed compound as a colorless oily substance (yield: 443 mg, 1.25 mmol, 52.5%, 3 steps). TLC Rf=0.43 (n-hexane/ethyl acetate=1/1)

### (2) Synthesis of 2-(S)-2-(benzyloxycarbonyl)amino-N-(tert-butoxycarbonyl)-N-(2-tert-butyldimethylsilyloxyethyl)propylamine

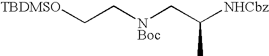

2-(S)-2-(Benzyloxycarbonyl)amino-N-(tert-butoxycarbonyl)-N-(2-hydroxyethyl)propylamine (443 mg, 1.25 mmol) was dissolved in dichloromethane (3 mL), and imidazole (128 mg, 1.88 mmol) and tert-butyldimethylsilyl chloride (283 mg, 1.88 mmol) were added to the solution, followed by stirring at room temperature for 1.5 hours. A saturated aqueous ammonium chloride solution was added to the solution, which was then extracted with ethyl acetate. The organic layer was washed with saturated brine, dried over anhydrous sodium sulfate and concentrated under vacuum to give the aimed compound as a colorless oily substance (yield: 480 mg, 1.02 mmol, 82.2%). TLC Rf=0.87 (n-hexane/ethyl acetate=1/1)

### (3) Synthesis of 2-(S)-2-amino-N-(tert-butoxycarbonyl)-N-(3-tert-butyldimethylsilyloxypropyl)propylamine

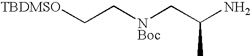

2-(S)-2-(Benzyloxycarbonyl)amino-N-(tert-butoxycarbonyl)-N-(2-tert-butyldimethylsilyloxyethyl)propylamine (490 mg, 1.04 mmol) was dissolved in methanol (3 mL). Catalytic reduction was carried out in the presence of 10% palladium-activated carbon (49.0 mg) under hydrogen flow. After completion of the reaction, dichloromethane was added to the mixture, which was filtered with Celite, and washed with ethyl acetate to remove the catalyst. The filtrate was concentrated under vacuum to give the aimed compound as a crude product (308 mg). This product was used in the subsequent reaction directly without purification.

### (4) Synthesis of 2-(S)-1-(4-bromoisoquinoline-5-sulfonyl)-N-(tert-butoxycarbonyl)-N-(2-tert-butyldimethylsilyloxyethyl)propylamine

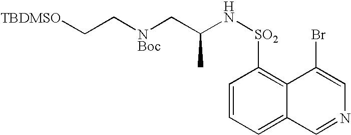

2-(S)-2-Amino-N-(tert-butoxycarbonyl)-N-(2-tert-butyldimethylsilyloxyethyl)propylamine (300 mg) was dissolved in dichloromethane (4 mL), and the resulting solution was cooled down to 0° C. Triethylamine (180 μL, 1.29 mmol), a catalytic amount of 4-dimethylaminopyridine, and 4-bromoisoquinoline-5-sulfonyl chloride (339 mg, 1.10 mmol) were subsequently added to the solution, which was then stirred at room temperature for 11 hours. Distilled water was added to the solution, which was then extracted with dichloromethane. The organic layer was washed with distilled water and dried over anhydrous sodium sulfate, followed by vacuum concentration. The residue was purified by silica gel column chromatography (n-hexane/acetone=4/1) to give the aimed compound as an orange oily substance (yield: 383 mg, 0.635 mmol, 57.7%). TLC Rf=0.39 (n-hexane/acetone=3/1)

### (5) Synthesis of 2-(S)-1-(4-bromoisoquinoline-5-sulfonyl)-N-(tert-butoxycarbonyl)-N-(2-hydroxyethyl)propylamine

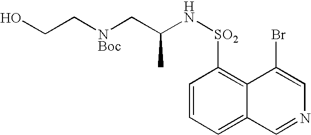

2-(S)-1-(4-Bromoisoquinoline-5-sulfonyl)-N-(tert-butoxy carbonyl)-N-(2-tert-butyldimethylsilyloxyethyl)propylamine (383 mg, 0.635 mmol) was dissolved in anhydrous THF (5 mL), and 1.0 M tetrabutylammonium fluoride (1.0 mL, 1.00 mmol) was added to the solution, which was then stirred at room temperature for 16 hours. A saturated aqueous ammonium chloride solution and dichloromethane were added to the solution, and the organic layer was washed with saturated ammonium chloride three times. The aqueous layer was then extracted with dichloromethane, and the organic layer was dried over anhydrous sodium sulfate, followed by vacuum concentration. The residue was purified by silica gel column chromatography (dichloromethane/methanol=30/1) to give the aimed compound as a colorless oily foam (yield: 260 mg, 0.532 mmol, 83.8%). TLC Rf=0.31 (dichloromethane/methanol=9/1)

### (6) Synthesis of (S)-1-(4-bromoisoquinoline-5-sulfonyl)-4-(tert-butoxycarbonyl)-2-methylpiperazine

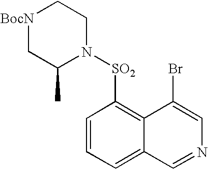

Under an argon atmosphere, 2-(S)-1-(4-bromoisoquinoline-5-sulfonyl)-N-(tert-butoxycarbon yl)-N-(2-hydroxyethyl)propylamine (260 mg, 0.532 mmol) was dissolved in anhydrous THF (4 mL), and the resulting solution was cooled down to 0° C. Triphenylphosphine (279 mg, 1.06 mmol) and diisopropyl azodicarbonate (208 μL, 1.06 mmol) were added to the solution, which was then stirred at room temperature for 23 hours. The solution was concentrated under vacuum, and the residue was purified by silica gel column chromatography (hexane/acetone=3/1 to 5/2) to give the aimed compound as a crude product in a white foam (387 mg). TLC Rf=0.65 (n-hexane/acetone=1/1)

### (7) Synthesis of (S)-1-(4-bromoisoquinoline-5-sulfonyl)-2-methylpiperazine dihydrochloride

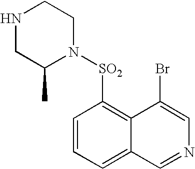

(S)-1-(4-Bromoisoquinoline-5-sulfonyl)-4-(tert-butoxycarbonyl)-2-methylpiperazine (238 mg) was dissolved in chloroform (1 mL), and a 1,4-dioxane solution of 4M hydrochloric acid (3.0 mL, 12.0 mmol) was added to the solution. After completion of the reaction, precipitated crystals were collected and washed with dichloromethane on a funnel, followed by drying under reduced pressure to give the aimed compound as a white solid.

(yield: 52.5 mg, 0.118 mmol, 36.0%, 3 steps) ¹H-NMR (400 MHz, DMSO-d₆) d 1.21 (d, 3H, J=6.0 Hz, CH₃), 2.93-2.96 (m, 1H, CH), 3.18-3.22 (m, 3H, CH, CH₂), 3.41-3.51 (m, 2H, CH₂), 4.27 (bs, 1H, CH), 7.93 (t, 1H, J=7.5 Hz, aromatic), 8.56 (d, 1H, J=7.5 Hz, aromatic), 8.61 (d, 1H, J=7.5 Hz, aromatic), 8.97 (s, 1H, aromatic), 9.46 (s, 1H, aromatic).

### Synthesis of (S)-hexahydro-1-(4-bromoisoquinoline-5-sulfonyl)-2-methyl-1H-1,4-diazepine dihydrochloride (hereinafter referred to as compound B7)

### (1) Synthesis of 2-(S)-2-(benzyloxycarbonyl)amino-N-(tert-butoxycarbonyl)-N-(3-hydroxypropyl)propylamine

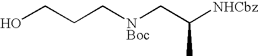

2-(S)-2-(Benzyloxycarbonyl)amino-1-propanol (2.99 g, 14.2 mmol) was dissolved in chloroform (30 mL), and the resulting solution was cooled down to 0° C. Triethylamine (3.0 mL, 21.5 mmol) and methane sulfonyl chloride (1.66 mL, 21.5 mmol) were added to the solution, and the mixture was stirred at room temperature for one hour. Distilled water was added to the solution, which was then extracted with chloroform. The organic layer was washed with saturated brine and dried over anhydrous sodium sulfate, followed by vacuum concentration. The resulting crystals were dried to give a crude product (4.70 g). The crude product (2.35 g) was then weighed and dissolved in THF (14 mL). 3-Amino-1-propanol (2.70 g, 35.9 mmol) was added to the resulting solution, which was then stirred at 80° C. for 24 hours. After the mixture was brought back to room temperature, distilled water was added to the solution, which was then extracted with dichloromethane. The organic layer was washed with distilled water and dried over anhydrous sodium sulfate, followed by vacuum concentration. The residue was then dissolved in dichloromethane (16 mL) and the resulting solution was cooled down to 0° C. Triethylamine (1.25 mL, 9.00 mmol) and di-tert-butyl dicarbonate (1.96 g, 9.00 mmol) were added to the solution, and the mixture was stirred at room temperature for one hour. Distilled water was added to the solution, which was then extracted with ethyl acetate. The organic layer was dried over anhydrous sodium sulfate, followed by vacuum concentration. The residue was purified by silica gel column chromatography (developing solvent: n-hexane/ethyl acetate=2/3) to give the aimed compound as a light yellow oily substance (yield: 1.74 g, 4.74 mmol, 66.8%, 3 steps). TLC Rf=0.23 (n-hexane/ethyl acetate=1/1)

### (2) Synthesis of 2-(S)-2-(benzyloxycarbonyl)amino-N-(tert-butoxycarbonyl)-N-(3-tert-butyldimethylsilyloxypropyl)propylamine

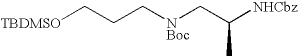

2-(S)-2-(Benzyloxycarbonyl)amino-N-(tert-butoxycarbonyl)-N-(3-hydroxypropyl)propylamine (1.74 g, 4.74 mmol) was dissolved in dichloromethane (6 mL), and imidazole (484 mg, 7.10 mmol) and tert-butyldimethylsilyl chloride (785 mg, 5.20 mmol) were added to the solution, followed by stirring at room temperature for 30 minutes. A saturated aqueous ammonium chloride solution was added to the solution, which was extracted with ethyl acetate. The organic layer was washed with saturated brine, dried over anhydrous sodium sulfate, and concentrated under vacuum to give the aimed compound as a colorless oily substance (2.16 g, 4.51 mmol, 95.1%). TLC Rf=0.83 (n-hexane/ethyl acetate=1/1)

### (3) Synthesis of 2-(S)-2-amino-N-(tert-butoxycarbonyl)-N-(3-tert-butyldimethylsilyloxypropyl)propylamine

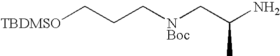

2-(S)-2-(Benzyloxycarbonyl)amino-N-(tert-butoxycarbonyl)-N-(3-tert-butyldimethylsilyloxypropyl)propylamine (2.16 g, 4.51 mmol) was dissolved in methanol (6 mL). Catalytic reduction was carried out in the presence of 10% palladium-activated carbon (207 mg) under hydrogen flow. After completion of the reaction, dichloromethane was added to the mixture, which was then filtered with Celite and washed with ethyl acetate to remove the catalyst. The filtrate was concentrated under vacuum to give the aimed compound as a colorless oily substance (1.41 g, 4.07 mmol, 90.3%). This substance was used in the subsequent reaction directly without purification.

### (4) Synthesis of 2-(S)-1-(4-bromoisoquinoline-5-sulfonyl)-N-(tert-butoxycarbonyl)-N-(3-tert-butyldimethylsilyloxypropyl)propylamine

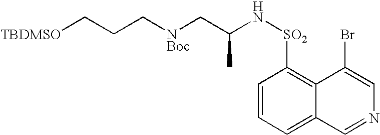

2-(S)-2-Amino-N-(tert-butoxycarbonyl)-N-(3-tert-butyldimethylsilyloxypropyl)propylamine (758 mg, 2.18 mmol) was dissolved in dichloromethane (5 mL), and the resulting solution was cooled down to 0° C. Triethylamine (304 μL, 2.18 mmol), a catalytic amount of 4-dimethylaminopyridine, and 4-bromoisoquinoline-5-sulfonyl chloride (613 mg, 2.00 mmol) were subsequently added to the solution, which was then stirred at room temperature for 14 hours. Distilled water was added to the solution, which was then extracted with dichloromethane. The organic layer was washed with distilled water and dried over anhydrous sodium sulfate, followed by vacuum concentration. The residue was purified by silica gel column chromatography (dichloromethane/methanol=40/1) to give the aimed compound as an orange oily substance (879 mg, 1.42 mmol, 71.2%). TLC Rf=0.50 (dichloromethane/methanol=18/1)

### (5) Synthesis of 2-(S)-1-(4-bromoisoquinoline-5-sulfonyl)-N-(tert-butoxycarbon yl)-N-(3-hydroxypropyl)propylamine

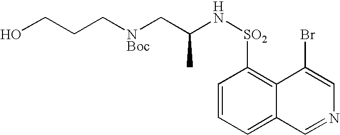

2-(S)-1-(4-Bromoisoquinoline-5-sulfonyl)-N-(tert-butoxy carbonyl)-N-(3-tert-butyldimethylsilyloxypropyl)propylamine (879 mg, 1.42 mmol) was dissolved in anhydrous THF (10 mL), and 1.0 M tetrabutylammonium fluoride (2.1 mL, 2.10 mmol) was added to the solution, which was then stirred at room temperature for 12 hours. A saturated aqueous ammonium chloride solution and dichloromethane were added to the solution, and the organic layer was washed with saturated ammonium chloride three times. The aqueous layer was then extracted with dichloromethane, and the organic layer was dried over anhydrous sodium sulfate, followed by vacuum concentration. The residue was purified by silica gel column chromatography (dichloromethane/methanol=50/1 to 40/1) to give the aimed compound as a colorless oily foam (618 mg, 1.23 mmol, 86.6%). TLC Rf=0.43 (dichloromethane/methanol=9/1)

### (6) Synthesis of (S)-hexahydro-1-(4-bromoisoquinoline-5-sulfonyl)-4-(tert-butoxycarbonyl)-2-methyl-1H-1,4-diazepine

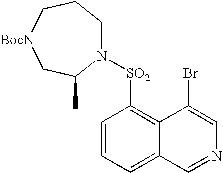

Under an argon atmosphere, 2-(S)-1-(4-bromoisoquinoline-5-sulfonyl)-N-(tert-butoxycarbon yl)-N-(3-hydroxypropyl)propylamine (618 mg, 1.23 mmol) was dissolved in anhydrous THF (11 mL), and the resulting solution was cooled down to 0° C. Triphenylphosphine (483 mg, 1.84 mmol) and diisopropyl azodicarbonate (362 μL, 1.84 mmol) were added to the solution, followed by stirring at room temperature for 23 hours. The solution was concentrated under vacuum, and the residue was purified by silica gel column chromatography (hexane/acetone=3/1 to 3/2) to give the aimed compound as a white foam (537 mg, 1.10 mmol, 90.1%). TLC Rf=0.37 (n-hexane/acetone=2/1)

### (7) Synthesis of 2-(S)-hexahydro-2-methyl-1-(4-bromoisoquinoline-5-sulfonyl)-1H-1,4-diazepine dihydrochloride

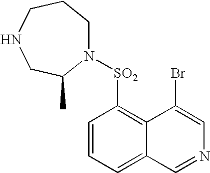

(S)-Hexahydro-1-(4-bromoisoquinoline-5-sulfonyl)-4-(tert-butoxycarbonyl)-2-methyl-1H-1,4-diazepine (100 mg, 0.206 mmol) was dissolved in chloroform (1 mL), and a 1,4-dioxane solution of 4M hydrochloric acid (1.5 mL, 6.00 mmol) was added to the solution. After completion of the reaction, precipitated crystals were collected and washed with dichloromethane on a funnel to give the aimed compound as a white solid. (90.6 mg, 0.198 mmol, 96.1%) ¹H-NMR (400 MHz, DMSO-d₆) d 1.17 (d, 3H, J=6.4 Hz, CH₃), 1.95-2.10 (m, 2H, CH₂), 3.00-3.10 (m, 1H, CH), 3.17-3.29 (m, 2H, CH₂), 3.45-3.48 (m, 1H, CH), 3.59-3.68 (m, 2H, CH₂), 4.35-4.50 (m, 1H, CH), 7.89 (t, 1H, J=7.9 Hz, aromatic), 8.25 (d, 1H, J=7.9 Hz, aromatic), 8.50 (d, 1H, J=7.9 Hz, aromatic), 8.97 (s, 1H, aromatic), 9.46 (s, 1H, aromatic).

### Synthesis of (S)-octahydro-1-(4-bromoisoquinoline-5-sulfonyl)-2-methyl-1,4-diazocine dihydrochloride (hereinafter referred to as compound B8)

2-(S)-2-Amino-N-(tert-butoxycarbonyl)-N-(4-tert-butyldimethylsilyloxybutyl)propylamine was synthesized following the synthesis steps (1) to (3) for the compound F8.

### (4) Synthesis of 2-(S)-1-(4-bromoisoquinoline-5-sulfonyl)-N-(tert-butoxycarbonyl)-N-(4-tert-butyldimethylsilyloxybutyl)propylamine

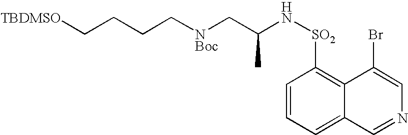

2-(S)-2-Amino-N-(tert-butoxycarbonyl)-N-(4-tert-butyldimethylsilyloxybutyl)propylamine (468 mg, 1.30 mmol) was dissolved in dichloromethane (3 mL), and the resulting solution was cooled down to 0° C. Triethylamine (181 μL, 1.28 mmol), acatalytic amount of 4-dimethylaminopyridine, and 4-bromoisoquinoline-5-sulfonyl chloride (363 mg, 1.18 mmol) were subsequently added to the solution, a which was then stirred at room temperature for 16 hours. Distilled water was added to the solution, which was then extracted with dichloromethane. The organic layer was washed with distilled water and dried over anhydrous sodium sulfate, followed by vacuum concentration. The residue was purified by silica gel column chromatography (dichloromethane/methanol=40/1) to give the aimed compound as an orange oily substance (yield: 522 mg, 0.827 mmol, 70.1%). TLC Rf=0.73 (dichloromethane/methanol=9/1)

### (5) Synthesis of 2-(S)-1-(4-bromoisoquinoline-5-sulfonyl)-N-(tert-butoxycarbon yl)-N-(4-hydroxybutyl)propylamine

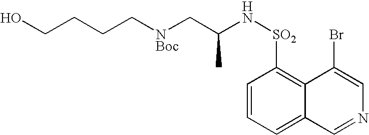

2-(S)-1-(4-Bromoisoquinoline-5-sulfonyl)-N-(tert-butoxy carbonyl)-N-(4-tert-butyldimethylsilyloxybutyl)propylamine (522 mg, 0.827 mmol) was dissolved in anhydrous THF (6 mL), and 1.0 M tetrabutylammonium fluoride (1.4 mL, 1.40 mmol) was added to the solution, which was then stirred at room temperature for 22 hours. A saturated aqueous ammonium chloride solution and dichloromethane were added to the solution, and the organic layer was washed with saturated ammonium chloride three times. The aqueous layer was then extracted with dichloromethane, and the organic layer was dried over anhydrous sodium sulfate, followed by vacuum concentration. The residue was purified by silica gel column chromatography (dichloromethane/methanol=35/1 to 30/1) to give the aimed compound as a colorless oily foam (yield: 271 mg, 0.524 mmol, 63.4%). TLC Rf=0.37 (dichloromethane/methanol=9/1)

### (6) Synthesis of (S)-octahydro-1-(4-bromoisoquinoline-5-sulfonyl)-4-(tert-butoxycarbonyl)-2-methyl-1,4-diazocine

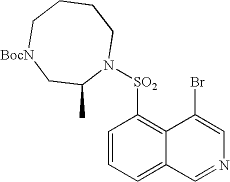

Under an argon atmosphere, 2-(S)-1-(4-chloroisoquinoline-5-sulfonyl)-N-(tert-butoxycarbonyl)-N-(3-hydroxypropyl)propylamine (271 mg, 0.524 mmol) was dissolved in anhydrous THF (5 mL), and the resulting solution was cooled down to 0° C. Triphenylphosphine (206 mg, 0.786 mmol) and diisopropyl azodicarbonate (154 μL, 0.786 mmol) were added to the solution, followed by stirring at room temperature for 12 hours. The solution was then concentrated under vacuum, and the residue was purified by silica gel column chromatography (n-hexane/acetone=3/1 to 5/2) to give the aimed compound as a crude product in a white foam (286 mg). TLC Rf=0.46 (n-hexane/acetone=2/1)

### (7) Synthesis of (S)-octahydro-1-(4-bromoisoquinoline-5-sulfonyl)-2-methyl-1,4-diazocine dihydrochloride

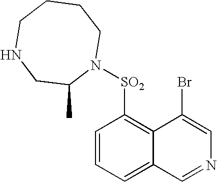

(S)-Octahydro-1-(4-bromoisoquinoline-5-sulfonyl)-4-(tert-butoxycarbonyl)-2-methyl-1,4-diazocine (140 mg) was dissolved in chloroform (1 mL), and a 1,4-dioxane solution of 4 M hydrochloric acid (2 mL, 8.00 mmol) was added to the solution. After completion of the reaction, precipitated crystals were collected and washed with dichloromethane on a funnel to give the aimed compound as a light yellow solid. (98.6 mg, 0.209 mmol, 81.7%) ¹H-NMR (400 MHz, DMSO-d₆) d 1.17 (d, 3H, J=6.8 Hz, CH₃), 1.67-1.94 (m, 4H, 2CH₂), 3.11-3.39 (m, 4H, 2CH₂), 3.11-3.39 (m, 4H, 2CH₂), 3.66 (bs, 2H, CH₂), 4.25 (bs, 1H, CH), 7.90 (t, 1H, J=7.9 Hz, aromatic), 8.28 (d, 1H, J=7.9 Hz, aromatic), 8.51 (d, 1H, J=7.9 Hz, aromatic), 8.96 (s, 1H, aromatic), 9.46 (s, 1H, aromatic).

### Synthesis of (S)-hexahydro-1-(4-chloroisoquinoline-5-sulfonyl)-2-methyl-1H-1,4-diazepine dihydrochloride (hereinafter referred to as compound C7)

2-(S)-2-Amino-N-(tert-butoxycarbonyl)-N-(3-tert-butyldimethylsilyloxybutyl)propylamine was synthesized following the synthesis steps (1) to (3) for the compound F7.

### (4) Synthesis of 2-(S)-1-(4-chloroisoquinoline-5-sulfonyl)-N-(tert-butoxycarbonyl)-N-(3-tert-butyldimethylsilyloxypropyl)propylamine

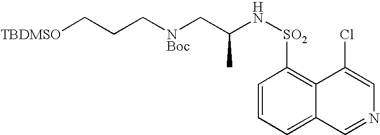

2-(S)-2-Amino-N-(tert-butoxycarbonyl)-N-(3-tert-butyldimethylsilyloxypropyl)propylamine (678 mg, 1.95 mmol) was dissolved in dichloromethane (4 mL), and the resulting solution was cooled to 0° C. Triethylamine (271 μL, 1.95 mmol), a catalytic amount of 4-dimethylaminopyridine, and 4-chloroisoquinoline-5-sulfonyl chloride (7) (468 mg, 1.78 mmol) were subsequently added to the solution, which was then stirred at room temperature for 21 hours. Distilled water was added to the solution, which was then extracted with dichloromethane. The organic layer was washed with distilled water and dried over anhydrous sodium sulfate, followed by vacuum concentration. The residue was purified by silica gel column chromatography (dichloromethane/methanol=35/1) to give the aimed compound as an orange oily substance (620 mg, 1.08 mmol, 60.8%). TLC Rf=0.43 (dichloromethane/methanol=18/1)

### (5) Synthesis of 2-(S)-1-(4-chloroisoquinoline-5-sulfonyl)-N-(tert-butoxycarbonyl)-N-(3-hydroxypropyl)propylamine

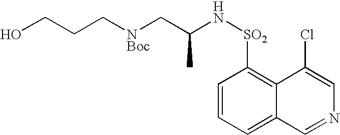

2-(S)-1-(4-Chloroisoquinoline-5-sulfonyl)-N-(tert-butoxycarbonyl)-N-(3-tert-butyldimethylsilyloxypropyl)propylamine (620 mg, 1.08 mmol) was dissolved in anhydrous THF (7 mL), and 1.0 M tetrabutylammonium fluoride (1.5 mL, 1.50 mmol) was added to the solution, which was then stirred at room temperature for 13 hours. A saturated aqueous ammonium chloride solution and dichloromethane were added to the solution, and the organic layer was washed with saturated ammonium chloride three times. The aqueous layer was then extracted with dichloromethane, and the organic layer was dried over anhydrous sodium sulfate, followed by vacuum concentration. The residue was purified by silica gel column chromatography (dichloromethane/methanol=100/1 to 50/1) to give the aimed compound as a colorless oily foam (428 mg, 0.934 mmol, 86.5%). TLC Rf=0.51 (dichloromethane/methanol=9/1)

### (6) Synthesis of (S)-hexahydro-4-(tert-butoxycarbonyl)-1-(4-chloroisoquinoline-5-sulfonyl)-2-methyl-1H-1,4-diazepine

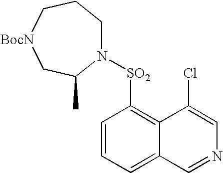

Under an argon atmosphere, 2-(S)-1-(4-chloroisoquinoline-5-sulfonyl)-N-(tert-butoxycarbonyl)-N-(3-hydroxypropyl)propylamine (419 mg, 0.914 mmol) was dissolved in anhydrous THF (8 mL), and the resulting solution was cooled down to 0° C. Triphenylphosphine (360 mg, 1.37 mmol) and diisopropyl azodicarbonate (270 μL, 1.37 mmol) were added to the solution, followed by stirring at room temperature for 18 hours. The solution was then concentrated under vacuum, and the residue was purified by silica gel column chromatography (hexane/acetone=3/1) to give the aimed compound as a white foam (390 mg, 0.886 mmol, 96.9%). TLC Rf=0.45 (n-hexane/acetone=2/1)

### (7) Synthesis of (S)-hexahydro-1-(4-chloroisoquinoline-5-sulfonyl)-2-methyl-1H-1,4-diazepine dihydrochloride

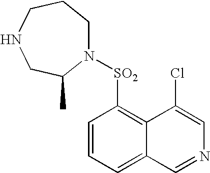

(S)-Hexahydro-4-(tert-butoxycarbonyl)-1-(4-chloroisoquinoline-5-sulfonyl)-2-methyl-1H-1,4-diazepine (150 mg, 0.340 mmol) was dissolved in chloroform (1.5 mL), and a 1,4-dioxane solution of 4M hydrochloric acid (2.0 mL, 8.00 mmol) was added to the solution. After completion of the reaction, precipitated crystals were collected and washed with dichloromethane on a funnel, followed by drying under reduced pressure to give the aimed compound as a white solid. (77.1 mg, 0.186 mmol, 54.7%) ¹H-NMR (400 MHz, DMSO-d₆) d 1.20 (d, 3H, J=6.8 Hz, CH₃), 1.96-2.06 (m, 2H, CH₂), 3.00-3.15 (m, 1H, CH), 3.18-3.35 (m, 2H, CH₂), 3.45-3.50 (m, 1H, CH), 3.55-3.71 (m, 2H, CH₂), 7.90 (t, 1H, J=7.7 Hz, aromatic), 8.26 (d, 1H, J=7.7 Hz, aromatic), 8.52 (d, 1H, J=7.7 Hz, aromatic), 8.79 (s, 1H, aromatic), 9.44 (s, 1H, aromatic).

### Synthesis of (S)-octahydro-1-(4-chloroisoquinoline-5-sulfonyl)-2-methyl-1,4-diazocine dihydrochloride (hereinafter referred to as compound C8)

2-(S)-2-Amino-N-(tert-butoxycarbonyl)-N-(4-tert-butyldimethylsilyloxybutyl)propylamine was synthesized following the synthesis steps (1) to (3) for the compound F8.

### (4) Synthesis of 2-(S)-1-(4-chloroisoquinoline-5-sulfonyl)-N-(tert-butoxycarbonyl)-N-(4-tert-butyldimethylsilyloxybutyl)propylamine

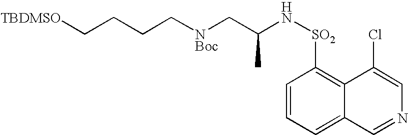

2-(S)-2-Amino-N-(tert-butoxycarbonyl)-N-(4-tert-butyldimethylsilyloxybutyl)propylamine (462 mg, 1.28 mmol) was dissolved in dichloromethane (3 mL), and the resulting solution was cooled down to 0° C. Triethylamine (178 μL, 1.28 mmol), a catalytic amount of 4-dimethylaminopyridine, and 4-chloroisoquinoline-5-sulfonyl chloride (286 mg, 1.09 mmol) were subsequently added to the solution, which was then stirred at room temperature for 16 hours. Distilled water was added to the solution, which was then extracted with dichloromethane. The organic layer was washed with distilled water and dried over anhydrous sodium sulfate, followed by vacuum concentration. The residue was purified by silica gel column chromatography (dichloromethane/methanol=40/1) to give the aimed compound as an orange oily substance. (yield: 511 mg, 0.871 mmol, 79.9%). TLC Rf=0.58 (dichlorome

### (5) Synthesis of 2-(S)-1-(4-chloroisoquinoline-5-sulfonyl)-N-(tert-butoxycarbonyl)-N-(4-hydroxybutyl)propylamine

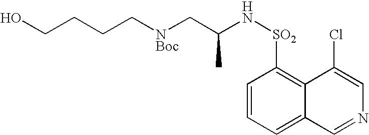

2-(S)-1-(4-Chloroisoquinoline-5-sulfonyl)-N-(tert-butoxycarbonyl)-N-(4-tert-butyldimethylsilyloxybutyl)propylamine (511 mg, 0.871 mmol) was dissolved in anhydrous THF (6 mL), and 1.0 M tetrabutylammonium fluoride (1.30 mL, 1.30 mmol) was added to the solution, which was then stirred at room temperature for 24 hours. A saturated aqueous ammonium chloride solution and dichloromethane were added to the solution, and the organic layer was washed with saturated ammonium chloride three times. The aqueous layer was then extracted with dichloromethane, and the organic layer was dried over anhydrous sodium sulfate, followed by vacuum concentration. The residue was purified by silica gel column chromatography (dichloromethane/methanol=35/1 to 30/1) to give the aimed compound as a colorless oily foam (yield: 369 mg, 0.776 mmol, 89.1%). TLC Rf=0.33 (dichloromethane/methanol=9/1)

### (6) Synthesis of (S)-octahydro-4-(tert-butoxycarbonyl)-1-(4-chloroisoquinoline-5-sulfonyl)-2-methyl-1,4-diazocine

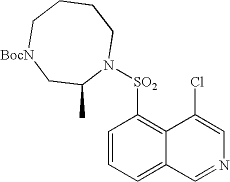

Under an argon atmosphere, 2-(S)-1-(4-chloroisoquinoline-5-sulfonyl)-N-(tert-butoxycarbonyl)-N-(4-hydroxybutyl)propylamine (369 mg, 0.776 mmol) was dissolved in anhydrous THF (7 mL). Triphenylphosphine (305 mg, 1.16 mmol) and diisopropyl azodicarbonate (228 μL, 1.16 mmol) were added to the solution, followed by stirring at room temperature for 15 hours. The solution was concentrated under vacuum and the residue was purified by silica gel column chromatography (n-hexane/acetone=3/1 to 5/2) to give the aimed compound as a crude product in a white foam (497 mg). TLC Rf=0.40 (n-hexane/acetone=2/1)

### (7) Synthesis of (S)-octahydro-1-(4-chloroisoquinoline-5-sulfonyl)-2-methyl-1,4-diazocine dihydrochloride

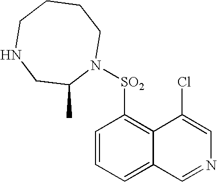

(S)-Octahydro-4-(tert-butoxycarbonyl)-1-(4-chloroisoquinoline-5-sulfonyl)-2-methyl-1,4-diazocine (109 mg) was dissolved in chloroform (1 mL), and a 1,4-dioxane solution of 4M hydrochloric acid (1.5 mL, 6.00 mmol) was added to the solution. After completion of the reaction, precipitated crystals were collected and washed with dichloromethane on a funnel, followed by drying under reduced pressure to give the aimed compound as a light yellow solid. (47.4 mg, 0.111 mmol, 48.4%) ¹H-NMR (400 MHz, DMSO-d₆) d 1.20 (d, 3H, J=6.8 Hz, CH₃), 1.96-2.06 (m, 2H, CH₂), 3.00-3.15 (m, 1H, CH), 3.18-3.35 (m, 2H, CH₂), 3.45-3.50 (m, 1H, CH), 3.55-3.71 (m, 2H, CH₂), 7.90 (t, 1H, J=7.7 Hz, aromatic), 8.26 (d, 1H, J=7.7 Hz, aromatic), 8.52 (d, 1H, J=7.7 Hz, aromatic), 8.79 (s, 1H, aromatic), 9.44 (s, 1H, aromatic).

### Example 2

### Measurement of Kinase Inhibitory Activity and Measurement of Intraocular Pressure

### ==Measurement of Kinase Inhibitory Activity==

Rho-kinase assays were performed using the compounds in various concentrations and the 50% inhibitory concentrations (hereinafter referred to as the “IC₅₀ value”) of the compounds for Rho-kinase were calculated. In the Rho-kinase assay, phosphorylation reaction was performed using an activated form of recombinant ROCK II (1-602) obtained by expressing human ROCK II cDNA in Sf9 cells as a substrate, and the phosphate incorporation activity was defined as the Rho-kinase activity. Namely, the compounds were each added to a reaction solution containing 50 mM Tris-HCL (ph 7.4), 10 μM MgCl₂, 2 mM EDTA, 2 mM EGTA, 0.1% tween 20, 50 μM RSK peptide, 10 μM [γ-³²P]ATP, and 10 nM ROCK II, and the reaction was allowed to proceed at 30° C. for 10 minutes. The reaction mixture was spotted on phosphocellulose paper, and the reaction was stopped by dipping the phosphocellulose paper into a phosphoric acid solution. The phosphocellulose paper was washed with the phosphoric acid solution, and then radioactivity was measured with a liquid scintillation counter.

Table 1 shows the IC₅₀ values for Rho-kinase of the individual compounds in the example and a reference compound (S)-hexahydro-1-(4-ethynylisoquinoline-5-sulfonyl)-2-methyl-1 H-1,4-diazepine dihydrochloride (hereinafter referred to as compound M).

TABLE 1

| Compound | IC₅₀ (μM) |
|---|---|
| F7 | 0.085 |
| F8 | 0.0391 |
| B6 | 0.634 |
| B7 | 0.0284 |
| B8 | 0.0485 |
| C7 | 0.0258 |
| C8 | 0.0211 |
| M | 0.0195 |

### ==Intraocular Pressure Measurement Experiments==

### (1) Preparation of Eye Drops

Eye drops were prepared by dissolving each compound (1.0 g) in a phosphate buffer solution which was prepared by dissolving 0.8 g of sodium dihydrogen phosphate and 0.5 g of sodium chloride in purified water such that the final weight was 100 g. The pH was adjusted to 7.0 with sodium hydroxide.

As a control, a phosphate buffer solution (pH 7.0) in which 1.0 g of the compound M was not dissolved was used.

### (2) Use in Rabbits

Each rabbit (NZW, male, 2.5 to 3.0 kg) was secured in a holder. One of the preparations (0.05 ml) was instilled into the right eye, and the phosphate buffer as control (0.05 ml) was instilled into the left eye. The intraocular pressures were measured serially using a TonoLab handheld mobile tonometer manufactured by Tiolat. The ratios to the control values were calculated and plotted with time (refer to figures).

### (3) Results

First, an experiment was conducted using the compound M as a conventional art. From 30 minutes to 5 hours after the instillation, a statistically significant intraocular-pressure-reducing effect was continuously observed relative to the control eye. At 120 minutes after the instillation, the intraocular-pressure-reducing effect was the maximum, the intraocular pressure being 28% lower than that of the control eye. In comparison, for the individual compounds, various intraocular-pressure-reducing characteristics were obtained.

In the right eye into which the compound F7 was instilled, from 30 minutes to 8 hours after the instillation, a statistically significant intraocular-pressure-reducing effect was continuously observed relative to the control eye. At 120 minutes after the instillation, the intraocular-pressure-reducing effect was the maximum, the intraocular pressure being 45% lower than that of the control eye. As is evident from the results, although the compound F7 had lower Rho-kinase inhibitory activity than the compound M, the intraocular-pressure-reducing effect in rabbits of the compound F7 was 1.9 times more than that of the compound M after 120 minutes from the instillation. Moreover, the period of time in which the intraocular-pressure-reducing effect was demonstrated in the compound F7 was about 3 hours longer than that for the compound M.

In the right eye into which the compound F8 was instilled, from 30 minutes to 6 hours after the instillation, a statistically significant intraocular-pressure-reducing effect was continuously observed relative to the control eye. At 120 minutes after the instillation, the intraocular-pressure-reducing effect was the maximum, the intraocular pressure being 45% lower than that of the control eye. Although the compound F8 had lower Rho-kinase inhibitory activity than the compound M, the intraocular-pressure-reducing effect in rabbits of the compound F8 was 1.6 times more than that of the compound M after 120 minutes from the instillation. Moreover, the period of time in which the intraocular-pressure-reducing effect was observed for the compound F8 was about 1 hour longer than that for the compound M.

In the right eye into which the compound B6 was instilled, from 30 minutes to 7 hours after the instillation, a statistically significant intraocular-pressure-reducing effect was continuously observed relative to the control eye. At 120 minutes after the instillation, the intraocular-pressure-reducing effect was the maximum, the intraocular pressure being 26% lower than that of the control eye. Although the compound B6 had lower Rho-kinase inhibitory activity than the compound M and the intensity of the intraocular-pressure-reducing effect in rabbits of the compound B6 was substantially the same as that of the compound M, the period of time in which the intraocular-pressure-reducing effect was observed for the compound B6 was about 2 hours longer than the compound M.

In the right eye into which the compound B7 was instilled, from 30 minutes to 10 hours after the instillation, a statistically significant intraocular-pressure-reducing effect was continuously observed relative to the control eye. At 120 minutes after the instillation, the intraocular-pressure-reducing effect was the maximum, the intraocular pressure being 56% lower than that of the control eye. Although the compound B7 had lower Rho-kinase inhibitory activity than the compound M, the intraocular-pressure-reducing effect in rabbits of the compound B7 was 2.0 times more than that of the compound M after 120 minutes from the instillation. Moreover, the period of time in which the intraocular-pressure-reducing effect was observed for the compound B7 was about 5 hours longer than that for the compound M.

In the right eye into which the compound B8 was instilled, from 30 minutes to 6 hours after the instillation, a statistically significant intraocular-pressure-reducing effect was continuously observed relative to the control eye. At 120 minutes after the instillation, the intraocular-pressure-reducing effect was the maximum, the intraocular pressure being 47% lower than that of the control eye. Although the compound B8 had lower Rho-kinase inhibitory activity than the compound M, the intraocular-pressure-reducing effect in rabbits of the compound B8 was 1.7 times more than that of the compound M after 120 minutes from the instillation. Moreover, the period of time in which the intraocular-pressure-reducing effect was observed for the compound B8 was about 1 hour longer than that for the compound M.

In the right eye into which the compound C6 was instilled, from 30 minutes to 8 hours after the instillation, a statistically significant intraocular-pressure-reducing effect was continuously observed relative to the control eye. At 120 minutes after the instillation, the intraocular-pressure-reducing effect was the maximum, the intraocular pressure being 54% lower than that of the control eye. Although the compound C6 had lower Rho-kinase inhibitory activity than the compound M, the intraocular-pressure-reducing effect in rabbits of the compound C6 was 1.9 times more than that of the compound M after 120 minutes from the instillation. Moreover, the period of time in which the intraocular-pressure-reducing effect was observed for the compound C6 was about 3 hours longer than that of the compound M.

In the right eye into which the compound C8 was instilled, from 30 minutes to 5 hours after the instillation, a statistically significant intraocular-pressure-reducing effect was continuously observed relative to the control eye. At 120 minutes after the instillation, the intraocular-pressure-reducing effect was the maximum, the intraocular pressure being 35% lower than that of the control eye. Although the compound C8 had lower Rho-kinase inhibitory activity than the compound M, the intraocular-pressure-reducing effect in rabbits of the compound C8 was 1.3 times more than that of the compound M after 120 minutes from the instillation. The period of time in which the intraocular-pressure-reducing effect was observed for the compound C8 was substantially the same as that for the compound M.

### (4) Conclusion

As is evident from the results, there was no significant positive correlation between the Rho-kinase inhibitory activity and the intraocular-pressure-reducing effect, and the intraocular-pressure-reducing characteristics vary depending on the compounds. Furthermore, the effect of the present invention was not influenced by the size of the ring of the cyclic aminosulfonyl group bonded to the position 5 of 4-haloisoquinoline.

### REFERENCE EXAMPLE

Using a reference compound (S)-hexahydro-1-(4-fluoroisoquinoline-5-sulfonyl)-4-glycyl-2-methyl-1H-1,4-diazepine dihydrochloride, the Rho-kinase inhibitory activity and the intraocular-pressure-reducing effect were measured.

### ==Measurement of Kinase Inhibitory Activity==

An Rho-kinase assay was performed, and the 50% inhibitory concentration (hereinafter referred to as the “IC₅₀ value”) was calculated. The IC₅₀ value of the reference compound (S)-hexahydro-1-(4-fluoroisoquinoline-5-sulfonyl)-4-glycyl-2-methyl-1H-1,4-diazepine dihydrochloride for Rho-kinase is shown in Table 2.

TABLE 2

| Compound | IC₅₀ (μM) |
|---|---|
| Reference Compound | 0.054 |

### ==Intraocular Pressure Measurement Experiments==

### (1) Preparation of Eye Drops

Eye drops were prepared by dissolving the compound (S)-hexahydro-1-(4-fluoroisoquinoline-5-sulfonyl)-4-glycyl-2-methyl-1H-1,4-diazepine dihydrochloride (1.0 g) in 1.0 g of dimethyl sulfoxide and adding the resulting solution to a boric acid solution prepared by dissolving 2.0 g of boric acid in purified water such that the final weight was 100 g. The pH was adjusted to 7.0 with sodium hydroxide.

### (2) Use in Rabbit

The eye drops were administered to a rabbit as in Example 1, and the intraocular pressures were measured. As a result, in the (S)-hexahydro-1-(4-fluoroisoquinoline-5-sulfonyl)-4-glycyl-2-methyl-1H-1,4-diazepine dihydrochloride, a statistically significant intraocular-pressure-reducing effect was not observed.

### (3) Conclusion

As described above, although the (S)-hexahydro-1-(4-fluoroisoquinoline-5-sulfonyl)-4-glycyl-2-methyl-1H-1,4-diazepine dihydrochloride had Rho-kinase inhibitory activity in the same level as that of the compounds used in the present invention, the reference compound did not have an intraocular-pressure-reducing effect. Consequently, it is evident that a compound having Rho-kinase inhibitory activity does not always have intraocular-pressure-reducing activity.

1. A method for treating glaucoma comprising administering to a glaucoma patient a compound represented by formula (1): 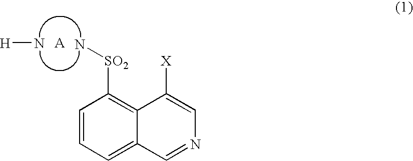
wherein ring A represents a 5- to 11-membered cyclic amino group, which may have a substituted group, and X represents a halogen.

2. A method for treating glaucoma comprising administering, in the form of eye drops, to a glaucoma patient a compound represented by formula (1): 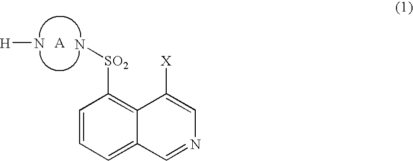
wherein ring A represents a 5- to 11-membered cyclic amino group, which may have a substituted group, and X represents a halogen.

3. A method for reducing intraocular pressure comprising administering to a glaucoma patient a compound represented by formula (1): 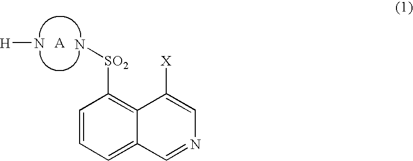
wherein ring A represents a 5- to 11-membered cyclic amino group, which may have a substituted group, and X represents a halogen.

4. The method for treating glaucoma according to claim 1, wherein the compound is represented by formula (2): 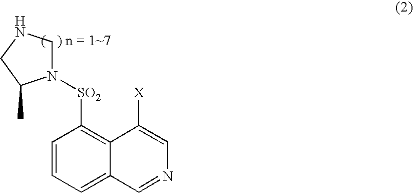
wherein X represents a halogen.

5. The method for treating glaucoma according to claim 2, wherein the compound is represented by formula (2): 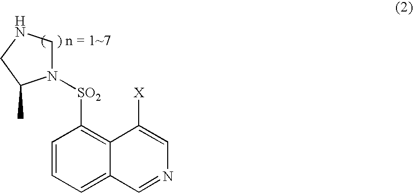
wherein X represents a halogen.

6. The method for reducing intraocular pressure according to claim 3, wherein the compound is represented by formula (2): 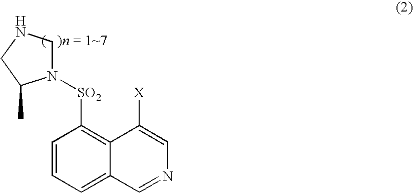
wherein X represents a halogen.

7. The method for treating glaucoma according to claim 1, wherein the compound is represented by any one of formulae (6) to (8): 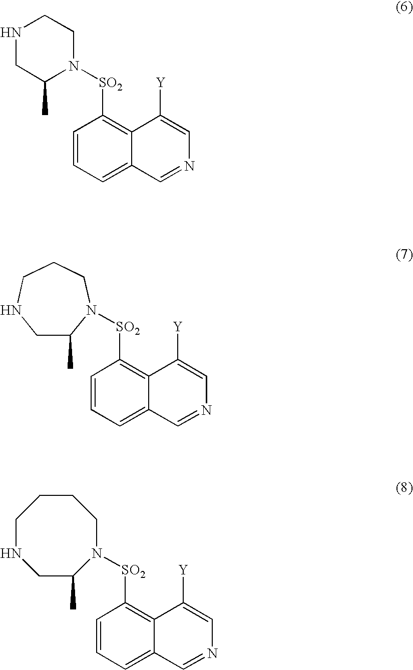
wherein Y represents Br, F, or Cl.

8. The method for treating glaucoma according to claim 2, wherein the compound is represented by any one of formulae (6) to (8): 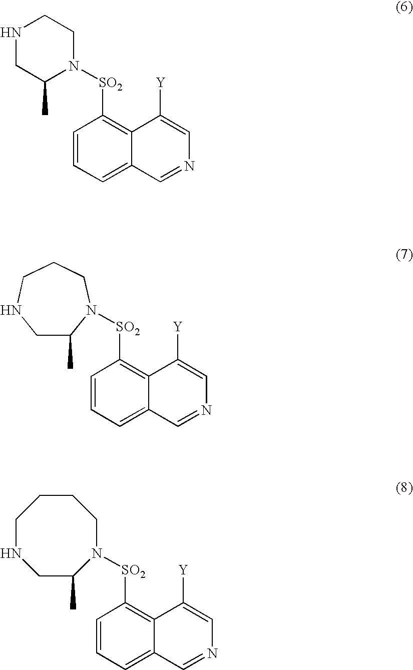
wherein Y represents Br, F, or Cl.

9. The method for reducing intraocular pressure according to claim 3, wherein the compound is represented by any one of formulae (6) to (8): 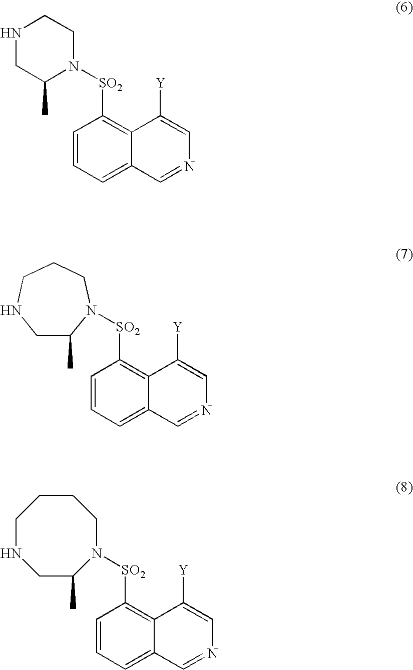
wherein Y represents Br, F, or Cl.
